(19) United States
(12) Patent Application Publication
(10) Pub. No.: US 2023/0383337 A1
(43) Pub. Date: Nov. 30, 2023

(54) ANALYSIS CARTRIDGE and ANALYSIS METHOD

(71) Applicant: Credo Diagnostics Biomedical Pte. Ltd., Singapore (SG)

(72) Inventors: Yu-Cheng Ou, New Taipei City (TW); Han-Yi Chen, New Taipei City (TW); Bing-Hsien Tsai, New Taipei City (TW); Kuan-Ying Chen, New Taipei City (TW); Chia-Chou Tseng, New Taipei City (TW)

(73) Assignee: Credo Diagnostics Biomedical Pte. Ltd., Singapore (SG)

(21) Appl. No.: 18/234,388

(22) Filed: Aug. 16, 2023

Related U.S. Application Data
(63) Continuation-in-part of application No. 17/545,956, filed on Dec. 8, 2021.

(30) Foreign Application Priority Data
Jun. 7, 2021 (TW) 110120577

(51) Int. Cl. C12Q 1/6844; B01L 3/00

(52) U.S. Cl. CPC C12Q 1/6844; C12Q 1/6806; B01L 3/502761

(57) ABSTRACT

An analysis cartridge and an analysis method, the analysis cartridge includes a first cover, a second cover, containers, a fluid tunnels and a rotary valve. The second cover has two opposite surfaces, a plurality of first through holes and a second through hole individually penetrate through the two opposite surfaces, and the first cover is attached to the second cover. The plurality of containers are disposed between the first cover and the second cover, with each of the containers being aligned to and filled in the first through holes. The plurality of the fluid tunnels is disposed on the first cover, and each of which is individually connected with a first pipette. The rotary valve is rotably disposed between the first cover and the second cover to correspond to the second through hole, and a flow channel disposed on the rotary valve is connected with the containers individually.

### CROSS REFERENCE TO RELATED APPLICATIONS

This application is a continuation-in-part of U.S. application Ser. No. 17/545,956, filed on Dec. 8, 2021. The content of the application is incorporated herein by reference.

### BACKGROUND OF THE INVENTION

### 1. Field of the Invention

The present disclosure generally relates to an analysis cartridge and an analysis method, and more particularly, to an analysis cartridge and analysis method for nucleic acid extraction and nucleic acid amplification.

### 2. Description of the Prior Art

Nucleic acid extraction and nucleic acid amplification are common technologies used in biomedical testing or diagnosis. Generally, a nucleic acid extraction kit or a nucleic acid extraction reagent are usually used in open and routine laboratories for nucleic acid extraction, followed by using a nucleic acid amplification kit or a nucleic acid amplification reagent to amplify specific nucleic acid fragments or detect specific nucleic acid fragments. However, the aforementioned kits or reagents are usually required manual operation, which is time-consuming and easy to result in contamination on samples or reagents, thereby being less efficiency in use on mass testing or production line mode testing.

Therefore, it is still necessary to the related arts to provide a novel and improved kit, reagent or device for nucleic acid extraction and nucleic acid amplification, so as to meet the practical requirements of the related arts.

### SUMMARY OF THE INVENTION

One of the objectives of the present disclosure provides an analysis cartridge, in which the connections between the rotary valve and each container may be controlled by rotating the rotary valve to a specific orientation through an external drive force, and then, samples, reagents, reaction solutions and other fluids may be transferred and mixed among the containers on demand with the volume thereof being precisely controlled as well, so as to facilitate the progress of each reaction step. The analysis cartridge of the present disclosure enables to provide an automatic testing process of sample-in result-out, thereby improving the limitations and poor efficacy of the routine laboratories and enhancing the testing efficiency and sensitivity.

Another one of the objectives of the present provides an analysis cartridge and an analysis method, in which at least one quantification chamber is additionally disposed within the analysis cartridge, to initiatively quantify the reagent either when absorbing the reagent from a container, or when injecting the reagent into the container. Accordingly, the analysis cartridge of the present disclosure enables to carry out the requested reaction such as the nucleic acid extraction and nucleic acid amplification in a more efficiency manner.

To achieve the purpose described above, one embodiment of the present disclosure provides an analysis cartridge including a main cover, at least one container, at least one pipette, at least one air pipe, and a rotary valve. The main cover has a first surface and a second surface opposite with each other. The first surface includes a first quantification chamber extending along a horizontal direction, at least one fluid tunnels, at least one gas tunnels and a storage chamber, wherein a first end of the first quantification chamber is connected to both the at least one fluid tunnel and the at least one gas tunnel, and a second end of the first quantification chamber is connected to the storage chamber. The at least one container is disposed on the second surface of the main cover, the at least one container overlapped the second end of the first quantification chamber in a vertical direction. The at least one pipette is disposed on the main cover and protruded from the second surface of the main cover, wherein the at least one pipette is connected to the second end of the first quantification chamber. The at least one air pipe is disposed on the main cover and protruded from the second surface of the main cover, wherein the at least one pipette and the at least one air pipe are disposed within the at least one container. The rotary valve is rotably disposed on the second surface of the first cover.

To achieve the purpose described above, one embodiment of the present disclosure provides an analysis cartridge including a main cover, at least one container, a first pipette, and a rotary valve. The main cover has a first surface and a second surface opposite with each other, the first surface comprising a first quantification chamber, a first fluid tunnel, a first gas tunnel and a storage chamber extending along horizontal directions, wherein a first end of the first quantification chamber is connected to the first fluid tunnel, a first end of the storage chamber is connected to the first gas tunnel, and a second end of the first quantification chamber is connected to a second end of the storage chamber. The container is disposed on the second surface of the main cover, and the container overlapped the second end of the first quantification chamber in a vertical direction. The first pipette is disposed on the main cover and protruded from the second surface of the main cover, wherein the first pipette is connected to the second end of the first quantification chamber. The rotary valve is rotably disposed on the second surface of the main cover.

To achieve the purpose described above, one embodiment of the present disclosure provides an analysis method including the following steps. Firstly, an analysis cartridge is provided and includes a main cover, a first pipette, a plurality of containers, and a rotary valve, wherein the main cover further includes a first quantification chamber, a first fluid tunnel, a first gas tunnel and a storage chamber extending along different horizontal directions, a first end of the first quantification chamber is connected to the first fluid tunnel, a first end of the storage chamber is connected to the first gas tunnel, and a second end of the first quantification chamber is connected to a second end of the storage chamber. The first pipette is connected to the second end of the first quantification chamber, wherein one of the containers is overlapped the second end of the first quantification chamber in a vertical direction and comprises a froze-dry sample disposed therein. Next, a reagent is injected into the first quantification chamber through the first end of the first quantification chamber. Then, a portion of the reagent is injected into the storage chamber through the second end of the first quantification chamber. Finally, the reagent within the first quantification chamber is transferred into the one of the containers through the rotary valve, to mix the froze-dry sample within the one of the containers.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 to FIG. 6 are schematic diagrams illustrating an analysis cartridge according to a first embodiment in the present disclosure, wherein:

FIG. 1 shows an exploded view of the analysis cartridge according to the first embodiment in the present disclosure;

FIG. 2 shows a top view of the analysis cartridge according to the first embodiment in the present disclosure;

FIG. 3 shows a cross-sectional view of a container of the analysis cartridge according to the first embodiment in the present disclosure;

FIG. 4 shows an exploded view of a rotary valve of the analysis cartridge according to the first embodiment in the present disclosure;

FIG. 5 shows a cross-sectional view of a pipette of the analysis cartridge according to the first embodiment in the present disclosure; and

FIG. 6 shows a cross-sectional view illustrating the usages of a short pulse laser beam to break cells in a fluid tunnel of the analysis cartridge according to the first embodiment in the present disclosure.

FIG. 7 to FIG. 10 are schematic diagrams illustrating an analysis cartridge according to a second embodiment in the present disclosure, wherein:

FIG. 7 shows an exploded view of the analysis cartridge according to the second embodiment in the present disclosure;

FIG. 8 shows a top view of the analysis cartridge according to the second embodiment in the present disclosure;

FIG. 9 shows an exploded view of a rotary valve of the analysis cartridge according to the second embodiment in the present disclosure; and

FIG. 10 shows a partial cross-sectional view of the rotary valve and a pipette of the analysis cartridge according to the second embodiment in the present disclosure.

FIG. 11 to FIG. 13 are schematic diagrams illustrating an analysis cartridge according to a third embodiment in the present disclosure, wherein:

FIG. 11 shows an exploded view of the analysis cartridge according to the third embodiment in the present disclosure;

FIG. 12 shows a exploded side view of the analysis cartridge according to the third embodiment in the present disclosure; and

FIG. 13 shows a side view of the analysis cartridge according to the third embodiment in the present disclosure.

FIG. 14 is a schematic diagram illustrating an analysis cartridge according to a fourth embodiment in the present disclosure.

### DETAILED DESCRIPTION

To provide a better understanding of the presented disclosure, preferred embodiments will be described in detail. The preferred embodiments of the present disclosure are illustrated in the accompanying drawings with numbered elements.

In the present disclosure, the formation of a first feature over or on a second feature in the description may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed. Furthermore, spatially relative terms, such as “beneath,” “below,” “lower,” “over,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. For example, if the device in the figures is turned over, elements described as “below” and/or “beneath” other elements or features would then be oriented “above” and/or “over” the other elements or features. The apparatus may be otherwise oriented (rotated 90 degrees or at other orientations) and the spatially relative descriptors used herein may likewise be interpreted accordingly.

It is understood that, although the terms first, second, third, etc. may be used herein to describe various elements, components, regions, layers and/or sections, these elements, components, regions, layers and/or sections should not be limited by these terms. These terms maybe only used to distinguish one element, component, region, layer and/or section from another region, layer and/or section. Terms such as “first,” “second,” and other numerical terms when used herein do not imply a sequence or order unless clearly indicated by the context. Thus, a first element, component, region, layer and/or section discussed below could be termed a second element, component, region, layer and/or section without departing from the teachings of the embodiments.

As disclosed herein, the term “about” or “substantial” generally means within 20%, preferably within 10%, and more preferably within 5%, 3%, 2%, 1%, or 0.5% of a given value or range. Unless otherwise expressly specified, all of the numerical ranges, amounts, values and percentages disclosed herein should be understood as modified in all instances by the term “about” or “substantial”. Accordingly, unless indicated to the contrary, the numerical parameters set forth in the present disclosure and attached claims are approximations that can vary as desired.

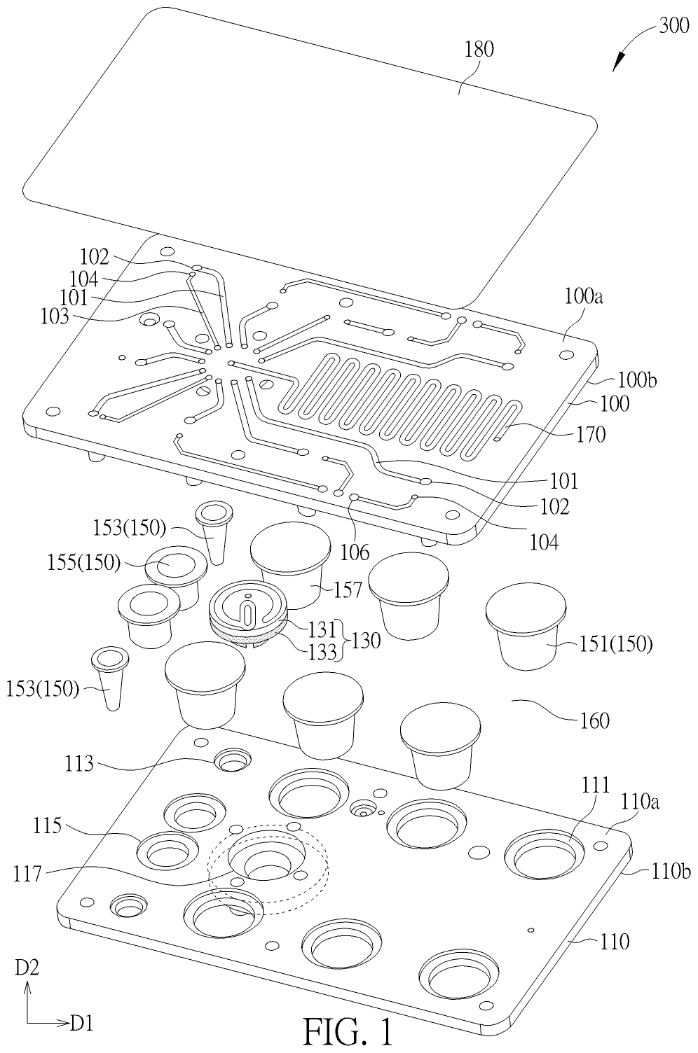

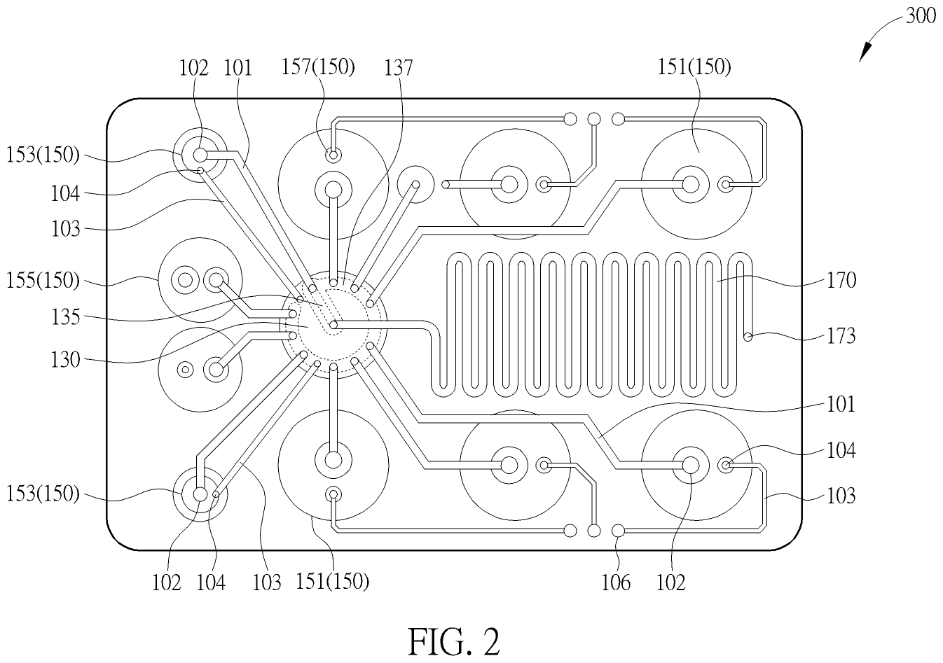

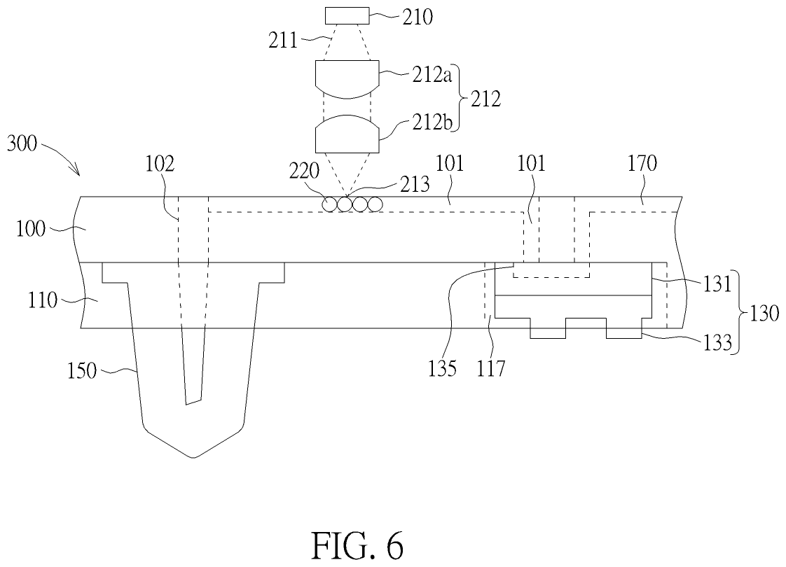

Please refers to FIG. 1 to FIG. 6, which illustrate an analysis cartridge 300 according to the first embodiment of the present disclosure, wherein FIG. 1 is a schematic diagrams of an exploded view of the analysis cartridge 300, FIG. 2 is a schematic diagram of a top view of the analysis cartridge 300, FIG. 6 is a schematic diagram of an operation of the analysis cartridge 300, and the rest drawings are schematic diagrams of a stereo view or a cross-sectional view showing the detailed components of the analysis cartridge 300. As shown in FIG. 1 and FIG. 2, the analysis cartridge 300 includes a first cover 100, a second cover 110 and a rotary valve 130. The first cover 100 for example includes two opposite surfaces, such as the first surface 100a and the second surface 100b as shown in FIG. 1, and the second cover 110 also includes two opposite surfaces, such as the first surface 110a and the second surface 110b as shown in FIG. 1. The second surface 100b of the first cover 100 faces to the first surface 110a of the second cover 110. While the analysis cartridge 300 is not yet assembled, the second cover 110 and the first cover 100 are separated from each other to define an accommodation space 160 (as shown in FIG. 1) therebetween, wherein the rotary valve 130, a plurality of containers 150 and other components may be disposed within the accommodation space 160. While assembling the analysis cartridge 300, the second surface 110b of the first cover 100 is attached to the first surface 110a of the second cover 110, and the rotary valve 130, the containers 150 and other components are all sandwiched between the second cover 110 and the first cover 100 with the accommodation space 160 being no longer existed, as shown in FIG. 2. In one embodiment, the first cover 100 and the second cover 110 are assemble for example through a thermal melting method or an ultrasonic method, so as to improve the reliability and malleability of the analysis cartridge 300, but not limited thereto.

Each of the first cover 100 and the second cover 110 for example includes a flat plate extending along a horizontal direction (such as the x-direction, as shown in the direction D1 in FIG. 1), and may be formed by a plastic injection molding method using the adequate material selected from the group including polypropylene (PP), polycarbonate (PC), polyimide (PI), polyethylene terephthalate (PET) and others having thermoplasticity and biocompatibility, but is not limited thereto. Also, the first cover 100 and the second cover 110 may have a mutually corresponding contour, for example, both are a rectangular shape, as shown in FIG. 1, but are not limited thereto. People skilled in the art should easily understand that the specific contour of the first cover 100 and the second cover 110 shown in FIG. 1 is only exemplary, and the first cover 100 and the second cover 110 may further include other applicable shapes based on practical product requirements.

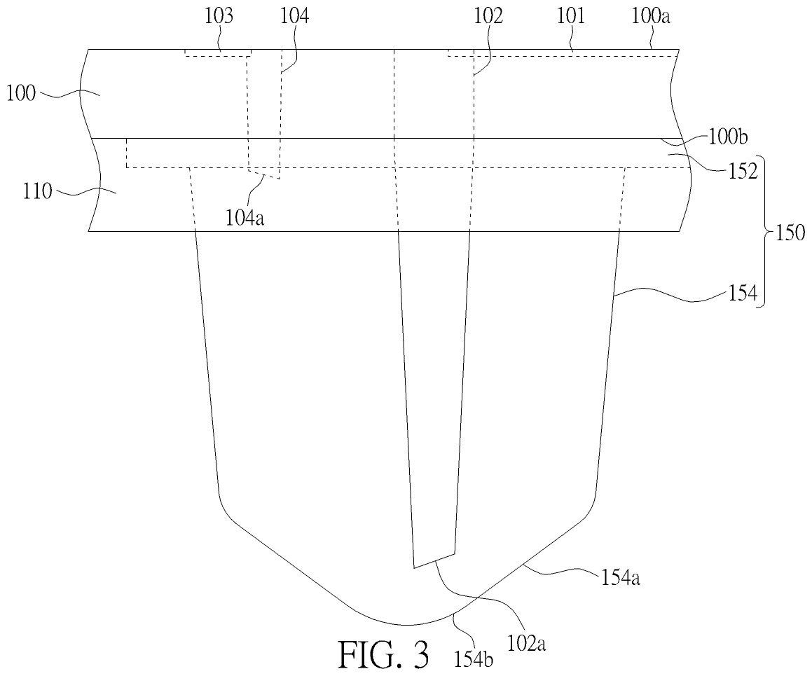

Precisely speaking, the first cover 100 further includes a plurality of fluid tunnels 101 and a plurality of gas tunnels 103 disposed on the first surface 100a. In the present embodiment, each of the fluid tunnels 101 and each of the gas tunnels 103 for example extends laterally along any direction which is parallel to the direction D1, to connect to a pipette 102 or a gas hole 104 for fluid circulation or gas circulation. One end of each gas tunnel 103 is connected to the gas hole 104, and the other end thereof is connected to a vent 106 disposed on the first cover 100 for exhausting air. Please also refers to FIG. 3, each of the pipettes 102 and each of the gas holes 104 are a hollow structure extended downwardly from the first surface 100a of the first cover 100 to protrude from the second surface 100b of the first cover 100. In one embodiment, the bottom portions of the pipette 102 and the gas hole 104 preferably include inclined sidewalls 102a, 104a respectively, as shown in FIG. 3, but not limited thereto. The inclined sidewall 102a of the pipettes 102 may improve the problem that liquid is easy to remain in the pipettes 102 while sucking liquid, and may also facilitate to punch through the sealing film during assembling. In another embodiment, the inclined sidewalls of the pipettes and the gas holes may also be optionally omitted (not shown in the drawings). Furthermore, due to the practical product requirements, the fluid tunnels and/or the gas tunnels may further have different extending directions, for example being extended along any direction which is perpendicular to the direction D1 (such as the direction D2), or are situated at different locations, and which is not limited to be the aforementioned types.

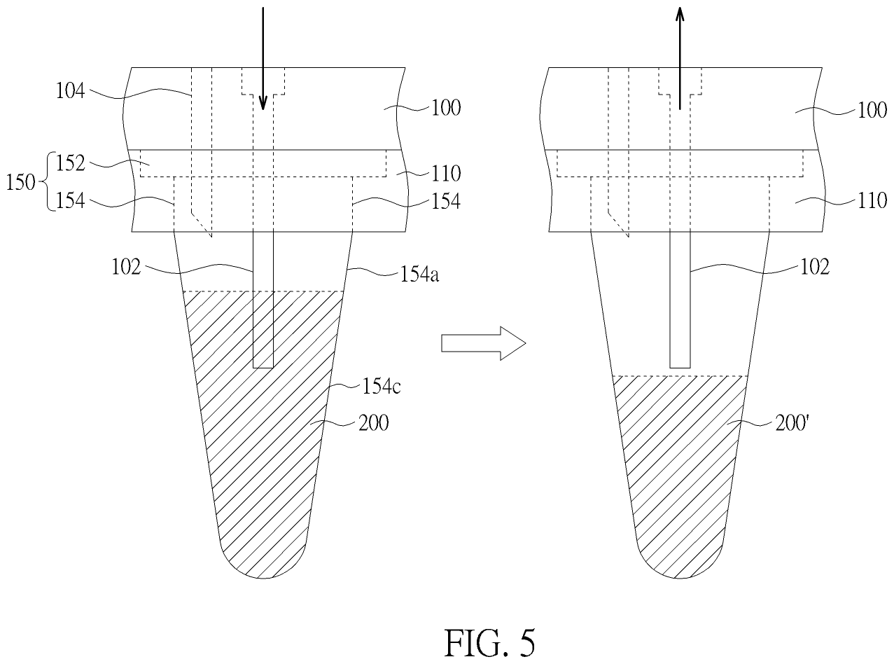

A plurality of through holes 111, 113, 115 are further disposed on the second cover 110, to penetrate through the first surface 110a and the second surface 110b sequentially, wherein each of the through holes 111, 113, 115 may have different sizes (e.g. different aperture sizes), so as to accommodate a plurality of containers 150 (e.g. the containers 151, 153, 155 as shown in FIG. 1 and FIG. 2) with different sizes, but not limited thereto. In other words, the practical size of each through hole may be diverse by the size of each container, and the practical size of each container may be diverse based on the actual product requirements, and which is not limited to those shown in FIG. 1 to FIG. 2, which may be easily understood by those skilled in the art. As shown in FIG. 3, each of the containers 150 includes a hollow main body 154 for accommodating various desired reagents based on practical product requirements, and the main body 154 is sealed by a film 152 for example including a material like aluminum foil or plastic. Preferably, the main body 154 includes an inclined portion 154a for facilitating to concentrate various reagents disposed within the container 150. The inclined portion 154a may include an inclined sidewall 154b, which is for example disposed at least at the bottom of the main body 154, as shown in FIG. 3, but not limited thereto. In another embodiment, the main body 154 may optionally include an inclined sidewall 154c as a whole, as shown in FIG. 5.

In one embodiment, the containers 150 for example include a plurality of reagent containers 151, at least one reaction container 153 and least one sample container 155, with each of the reagent containers 151 individually accommodating a cleaning reagent, a buffer, an eluent, a lysate or the like, with the at least one reaction container 153 accommodating various enzymes or reactants (such as primers or probes) for performing the reaction, and with the at least one sample container 155 accommodating various samples such as bacteria, cells or virus or samples suspected of carrying bacteria, cells or viruses and required the nucleic acid extraction and the nucleic acid amplification for confirmation. The quantity of the reaction containers 153 may be any suitable number, for example may be two as shown in FIG. 1. Then, the analysis cartridge 300 may perform different amplification and testing reaction at the same time through the two reaction containers 153, based on various primers and/or probes disposed therein, but is not limited thereto. People skilled in the art should easily understand that, in other embodiments, a single reaction container or more reaction containers may also be optionally disposed in the analysis cartridge, for achieving different testing requirements. In addition, the containers 150 may further include an extraction container 157 having a plurality of magnetic beads (not shown in the drawings) disposed therein, and the magnetic beads may be combined with the testing sample at the beginning of the testing for purification.

It is noted that, the pipettes 102 and the gas holes 104 disposed on the first cover 100 are in alignment with the through holes 111, 113, 115 disposed on the second cover 110, so that, the pipettes 102 and the gas holes 104 disposed on the first cover 100 may punch through the film 152 of each container 150 disposed within each through holes 111, 113, 115 by using the inclined sidewalls 102a, 104a thereof, during assembling the analysis cartridge 300, as shown in FIG. 3. Preferably, the pipettes 102 disposed on the first cover 100 may further extend into the bottom of the containers 150 after penetrating through the films 152 of the containers 150, more preferably, being extended to the portion closed to the inclined portion 154a; and the gas holes 104 disposed on the first cover 100 maybe located at the top portion of the containers 150, right located at the portion just penetrating through the films 152, as shown in FIG. 3, but not limited thereto.

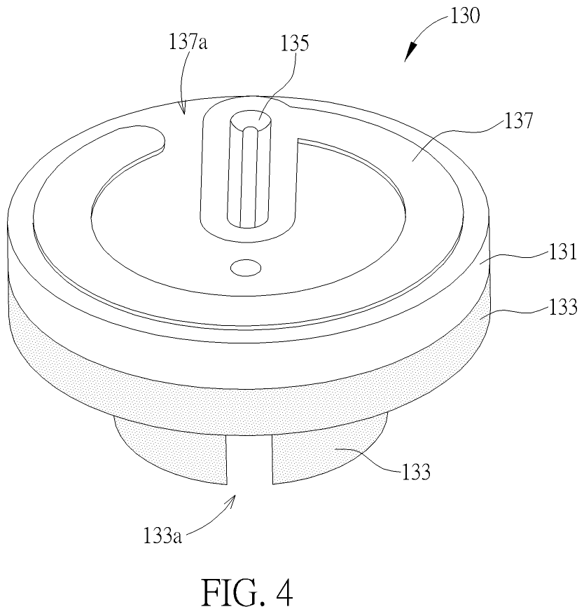

On the other hand, a through hole 117 is further disposed on the second cover 110, for accommodating the rotary valve 130 to rotate therein. Precisely speaking, the rotary valve 130 is for example consisted of a soft material in combined with a hard material, in order to improve the airtightness of the rotary valve 130 after being combined with the first cover 100 and the second cover 110. As shown in FIG. 4, the rotary valve 130 includes a first portion 131 and a second portion 133 stacked from top to bottom, wherein the first portion 131 for example includes thermoplastic polyurethanes (TPU), rubber, polyurethane material, polyethylene, polyethylene terephthalate (PET), thermoplastic polyester elastomer (TPEE), biocompatible resin, or a combination thereof, and the second portion 133 includes a rigid material different from that of the first portion 131, such as polypropylene fiber, polycarbonate, or the like, but not limited thereto. In this way, when the analysis cartridge 300 is assembled, the first portion 131 of the rotary valve 130 may be attached to the second surface 100b of the first cover 100, and the second portion 133 of the rotary valve 130 may be installed in the through hole 117, thereby achieving an airtight assembly manner.

In the present embodiment, the first portion 131 of the rotary valve 130 further includes a protrusion 137, with the protrusion 137 surrounding a flow channel 135 and forming an opening 137a, and the second portion 133 of the rotary valve 130 includes an engagement 133a. The flow channel 135 may include any suitable shape, for example the straight shape as shown in FIG. 4, but is not limited thereto. In this way, after the analysis cartridge 300 is assembled, the second portion 133 (including the engagement 133a) of the rotary valve 130 may be protruded into the through hole 117 of the second cover 110, to further externally connect to a motor (not shown in the drawings), with the motor driving and controlling the rotary valve 130 within the analysis cartridge 300 to rotate. In other words, the rotary valve 130 may be ratably disposed between the first cover 100 and the second cover 110. With such arrangements, one end of the flow channel 135 may be connected to different fluid tunnels 101 in sequence through the rotation of the rotary valve 130, when the opening 137a may be aligned to the gas holes 104 at the same time. While the rotary valve 130 further connects to a pump (not shown in the drawings) externally through a liquid temporary storage region 170, the various reagents within each container 150 may be sucked out, discharged, or transferred through a positive pressure or a negative pressure provided by the pump. In the present embodiment, the analysis cartridge 300 further includes the liquid temporary storage region 170 for example disposed on the first surface 100a of the first cover 100. As shown in FIG. 1 and FIG. 2, the liquid temporary storage region 170 may include a hollow tubular structure in a snaked shape or a continuously curved shape, wherein one end of the liquid temporary storage region 170 may be connected to another end of the flow channel 135, and another end of the liquid temporary storage region 170 may further include a pump connector 173 for externally connecting to the pump. Accordingly, the liquid temporary storage region 170 of the analysis cartridge 300 may be used to temporarily store the sucked-out reagent, so as to assist to suck, discharge or transfer the reagents.

Moreover, the analysis cartridge 300 may further include a flat film-shaped material (for example a sealing layer 180 as shown in FIG. 1) attached to the first surface 100a of the first cover 100 to seal the fluid tunnels 101, the gas tunnels 103 and the liquid temporary storage region 170 into closed channels.

In a preferably embodiment, the analysis cartridge 300 may be used in nucleic acid extraction and nucleic acid amplification, but is not limited thereto. For example, through rotating the rotary valve 130 to a specific orientation, the sample disposed within the sample container 155 maybe firstly transferred to one of the reagent containers 151 to rupture or to open the cells of the sample using a chemical method, followed by rotating the rotary valve 130 again to transfer the sample containing the ruptured or opened cells and the released substances thereof to the extraction container 157. The sample containing the ruptured or opened cells and the released substances thereof are combined with the magnetic beads within the extraction container 157 for purification. Then, the sample combined with the magnetic beads is further transferred to another reagent container 151 for washing, and finally, the desired biomaterial such as nucleic acid is eluted from the magnetic beads, for performing the subsequent testing. Subsequently, the biomaterial is also transferred to the reaction container 153 through the rotary valve 130 to carry out the desired reaction. If the reaction container 153 contains the lyophilized primer pair, nitrogenous bases and nucleic acid polymerase, and a polymerase chain reaction may be carried out after the biomaterial is injected into the reaction container 153, but the reaction is not limited thereto. In another embodiment, the reaction container 153 may optionally contain other enzymes or reagents, to carry out other reaction such as probe conjugation or enzymatic conjugation based on the product requirements. It is noted that, while transferring the aforementioned sample or biomaterial, the length of the pipettes 102 extended into each container 150 may be used to quantify the fluid. Precisely speaking, as shown in FIG. while a fluid (such as the aforementioned sample or biomaterial) 200 is injected into the container 150, the fluid 200 having an initial liquid level may cover the pipettes 102 to reach a specific height (as shown in the left panel of FIG. 5). Next, the fluid 200 is sucked out to result in the liquid level lowered and to leave the fluid 200′, and the bottom of the pipettes 102 may no longer be covered by the fluid 200′ (as shown in the right panel of FIG. 5) . Accordingly, the sucked-out volume of the fluid 200 may be accurately controlled, and it may be further confirmed using the volume of the fluid 200′ remained in the container 150. In other words, the specific liquid level is depended upon the desired volume of the fluid 200. When the larger volume of the fluid 200 to be sucked is desired, it may select the pipettes 102 that may extend into the container 150 deeper or the container 150 having a shorter length. When the smaller volume of the fluid 200 to be sucked is desired, it may select the pipettes 102 that may extend into the container 150 shallower (for example the pipette is extended into a half depth of the container 150 or is closed to the top of the container 150), or the container 150 having a longer length. In this way, the depth of the pipettes 102 extended into each container 150 may be adjusted according to the practical requirements of the testing, so as to quantify the transferred amount of the fluid.

Moreover, it is also noted that, while transferring the biomaterial to the reaction container 153 through the rotary valve 130, the rotary valve 130 is rotated to make the flow channel 135 thereof to align with the pipette 102 which is extended into the reaction container 153, and to make the opening 137a thereof to align with the gas hole 104 which is extended into the reaction container 153. Through these arrangements, the biomaterial maybe successfully injected into the reaction container 153 while the gas tunnel 103 is free for circulation. However, while a reaction is required to be performed in the reaction container 153, the rotary valve 130 may be rotated again to make the pipette 102 and the gas hole 104 which are extended into the reaction container 153 being no longer aligned with the flow channel 135 and the opening 137a. Then, the fluid tunnels 101 and the gas tunnels 103 may be closed thereby, so as to prevent the volume of the reactants and fluids disposed within the reaction container 153 from evaporation due to the increased temperature, or to prevent from condensation due to the decreased temperature, which may seriously affect the concentrations of the reactants and fluids. In other words, while the reaction is carried out in the reaction container 153, the pipette 102 and the gas hole 104 extended into the reaction container 153 may be covered by the protrusion 137 disposed on the rotary valve 130, so that the inner space of the reaction container 153 may reach an airtight state, thereby promoting the performance of the reaction.

Accordingly, in a preferable embodiment for nucleic acid extraction and nucleic acid amplification, the rotary valve 130 is rotated to communicate with the liquid temporary storage region 170 through the flow channel 135 thereon, and to communicate with the sample container 155 through the fluid tunnel 101. Meanwhile, the pump is driven to suck out the sample within the sample container 155 to the liquid temporary storage region 170. Next, the rotary valve 130 is rotated again to make one end of the flow channel 135 to communicate with the reagent container 151 (as shown in the upper right corner in FIG. 2) through the fluid tunnel 101, and to make the other end of the flow channel 135 to still communicate with the liquid temporary storage region 170, as the pump is driven to discharge and suck out the sample within the liquid temporary storage region 170 back and forth between the reagent container 151 and the liquid temporary storage region 170. Accordingly, the cells in the sample or the sample suspected to contain cells may be therefore ruptured or opened due to the lysis buffer disposed within the reagent container 151, as well as the physical force caused by the flow among the fluid tunnels 101, the flow channel 135 and the liquid temporary storage region 170, to obtain a first mixture by mixing the lysis buffer and the sample. Then, the rotary valve 130 is rotated again to make the flow channel 135 to communicate with the extraction container 137 through the fluid tunnel 101, with the first mixture temporarily stored in the liquid temporary storage region 170 being discharged into the extraction container 157 through the flow channel 135 and the fluid tunnel 101. The extraction container 157 contains magnetic beads whose surfaces have molecules for binding nucleic acids, and the magnetic beads may capture nucleic acids (if any) in the first mixture to form a nucleic acid-magnetic bead complex. Alternatively, the magnetic beads may not capture nucleic acids if there is no nucleic acid presented in the sample. Likewise, the magnetic beads are fully mixed with the first mixture to forma second mixture through the discharging and sucking out by the pump.

Next, the nucleic acid-magnetic bead complex (or only the magnetic beads if the nucleic acid does not exist) within the second mixture may be adsorbed by using a magnet or magnetic device (not shown in the drawings) placed outside the extraction container 157. The residue of the second mixture is then sucked out and transferred to the liquid temporary storage region 170, and the rotary valve 130 is next rotated to communicate with the used reagent container 151 (as shown in the upper right area of FIG. 2), to further transfer the residue of the second mixture from the liquid temporary storage region 170 to the used reagent container 151 for storage. Preferably, the magnet or the magnetic device is placed at a position far away from the inclined sidewall 102a of the pipette 102, so as to prevent the desired nucleic acid-magnetic bead complex from being sucked out from the extraction container 157 and discarded due to pumping suction.

After that, the rotary valve 130 is rotated again to connect to another reagent container 151 containing a cleaning reagent (for example the reagent container 151 disposed below the rotary valve 130 as shown in FIG. 2), and the magnet or the magnetic device is placed far away from the extraction container 157, thereby transferring the cleaning reagent to the liquid temporary storage region 170 and then to the extraction container 157. Accordingly, the nucleic acid-magnetic bead complex is released to mix with the cleaning reagent to form a third mixture. Then, the magnet or the magnetic device is placed again to adsorb the nucleic acid-magnetic bead complex, and the residue of the third mixture is transferred to the reagent container 151 (such as the reagent container 151 in the upper right area of FIG. 2) for storage.

When a buffer is applied, the nucleic acid-magnetic bead complex is also processed through the same steps in the aforementioned paragraph. People in the art should easily understand that, in another embodiment, the nucleic acid-magnetic bead complex may also be treated with the same or different cleaning reagents or buffer disposed in one or more reagent containers 151, so as to improve the extraction efficiency and the purity thereof.

Then, the rotary valve 130 is rotated again to communicate with another reagent container 151 containing an eluent (such the reagent container 151 in the lower right area in FIG. 2), and the magnet or the magnetic device is placed far away from the extraction container 157, followed by firstly transferring the eluent to the liquid temporary storage region 170 and then to the extraction container 157, wherein the eluent may break the bonding between the nucleic acid and the molecules on the surfaces of the magnetic beads, thereby releasing the nucleic acid. Then, the nucleic acid, the magnetic beads and the eluent may therefore form a fourth mixture. The magnet or the magnetic device is placed again to absorb the magnetic beads, and the residue of the fourth mixture (including the nucleic acid and the eluent) is then transferred to the liquid temporary storage region 170, and the rotary valve 130 is rotated again to communicate with the reaction container 153, the flow channel 135 and the liquid temporary storage region 170. It is noted that, the opening 137a formed by the semi-closed protrusion 137 of the rotary valve 130 is communicated with the reaction container 153 at this time through the gas tunnel 103 and the gas hole 104, and the residue of the fourth mixture (including the nucleic acid and the eluent) may be injected into the reaction container 153 from the liquid temporary storage region 170 as the gas tunnels 103 are free for circulation. On the other hand, while the reaction is performed within the reaction container 153, the rotary valve 130 is rotated to make the pipette 102 and the gas hole 104 extended into the reaction container 153 being not aligned with the flow channel 135 and the opening 137a, thereby blocking the fluid tunnel 101 and the gas tunnel 103.

In addition, the analysis cartridge 300 of the present disclosure enables to simultaneously carry out one or more acid amplification reactions, and an appropriate volume of the residue of the fourth mixture may be dispensed to two or more reaction containers 153. The nucleic acid contained in the residue of the fourth mixture is then amplified by an external instrument (not shown in the drawings) in the presence of a primer pair and/or a probe, deoxynucleoside triphosphate and polymerase, and the external instrument may further identify the sample contains a specific strain of bacteria or not by detecting the signal of the amplified nucleic acid.

In the aforementioned embodiment, cells within the sample are ruptured or opened by the lysis buffer disposed in the reagent container 151 and the physical force imposed back and forth between the flow channels 135, and the sample and the lysis buffer are mixed to form the first mixture, which then is further mixed with the magnetic beads in the extraction container 157 to form the nucleic acid-magnetic bead complex. In another improved embodiment, the sample and the lysis buffer may be transferred to the extraction container 157 individually, and mixed with magnetic beads to form the second mixture. Alternatively, the sample may be firstly mixed with the lysis buffer, and immediately transferred to the extraction container 157, thereby mixing with the magnetic beads to form the second mixture. Then, the second mixture may flow back and forth among the fluid tunnels 101, the flow channel 135 and the liquid temporary storage region 170, so that not only the cells in the second mixture are ruptured or opened due to the physical force and the lysis buffer, but also the nucleic acid released from the cells is captured by the magnetic beads during the mixing process, which may significantly reduce the time for nucleic acid extraction.

Through these arrangements, the analysis cartridge 300 according to the first embodiment of the present disclosure is provided. According to the present embodiment, the rotary valve 130 is rotably disposed in the analysis cartridge 300, and the external motor is linked with the rotary valve 130 in the analysis cartridge 300 to drive the rotary valve 130 to rotate to any orientation, so that, various fluids such as the sample, the reagents and the reactants disposed in each of the containers 150 may be freely transferred and mixed among the containers 150, and finally transferred to the reaction container 153 for carrying out the reaction. The rotary valve 130 includes the flow channel 135 and the opening 137a disposed thereon. While the sample, the reagents and the reactant are sucked out through the rotary valve 130, the rotary valve 130 is rotated to make the flow channel 135 and the opening 137a disposed thereon to align with the pipettes 102 and the gas holes 104 which are penetrated into the containers 150, respectively, so as to facilitate the transferring of fluids. On the other hand, while a reaction such as a nucleic acid extraction, a nucleic acid amplification, a cell rupture or cell opening reaction would be carried out in the containers 150, the rotary valve 130 is rotated to make the protrusion 137 thereon directly cover the pipette 102 and the gas hole 104 which are penetrated into the containers 150, thereby enabling the containers 150 to perform like an airtight state to prevent from contamination and to facilitate the reaction. With such arrangements, the analysis cartridge 300 of the present embodiment enables to provide an automated testing process of sample-in result-out, thereby improving the limitations and poor efficacy of the routine laboratories and enhancing the testing efficiency and sensitivity.

People in the art should also fully understand that the analysis cartridge of the present disclosure is not limited to the aforementioned type, and may include other examples or variations. For example, in the aforementioned embodiment, since the sample is processed chemically, a reagent container 151 containing reagent for rupturing or opening cell may be arranged in the analysis cartridge 300. However, in another embodiment, the cells may also be ruptured or opened through other methods such as a laser or an ultrasonic method, and devices for performing laser or ultrasonic cell disruption may be further arranged in the analysis cartridge and used together with an optical lens. For example, as shown in FIG. 6, a laser diode 210 maybe additionally provided, and a short pulse laser beam 211 emitted from the laser diode 210 may pass through an optical lens set 200 (including a light receiving lens 212a and a focusing lens 212b) and is focused on a focus 213. Then, the biomaterial flows between the liquid temporary storage region 170, the flow channel 135 of the rotary valve 130, the fluid tunnels 101, the pipettes 102, and the containers 151 may be irradiated by the short pulse laser beam 211 when passing through the focus 213, the cells 220 within the biomaterial maybe ruptured or opened to release the nucleic acid. However, in another embodiment, the laser diode, the optical lens set or the like may also be disposed in the analysis cartridge, or the optical lens set may be disposed in the analysis cartridge, with the laser diode being additionally provided for example on an instrument (not shown in the drawings) for accommodating the analysis cartridge.

The following description will detail the different embodiments of the analysis cartridge, and the following description will detail the dissimilarities among the different embodiments and the identical features will not be redundantly described. In order to compare the differences between the embodiments easily, the identical components in each of the following embodiments are marked with identical symbols.

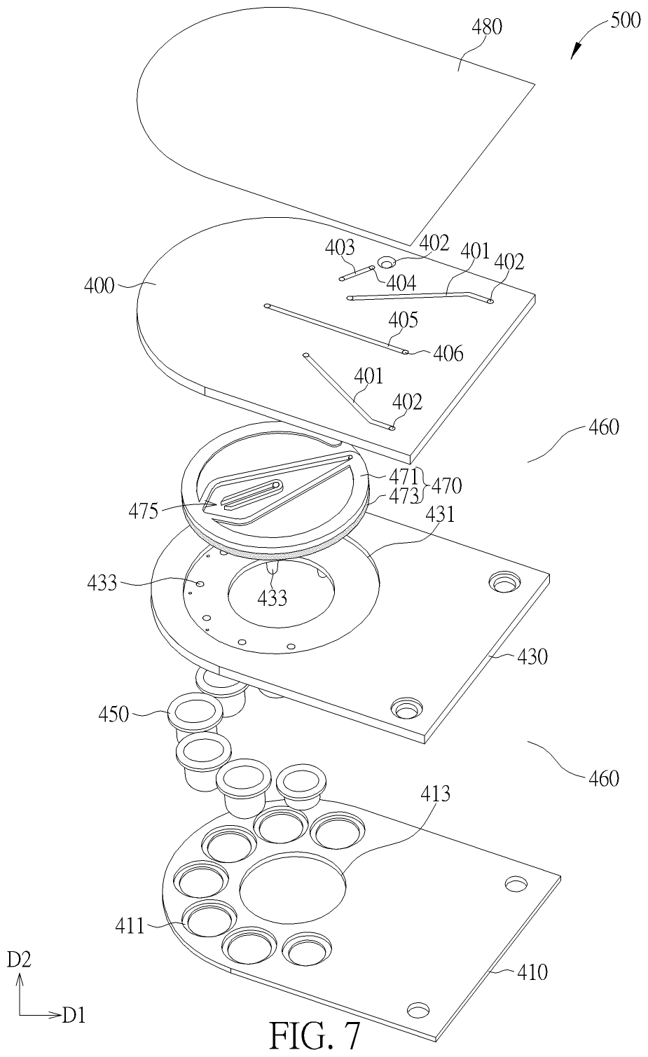

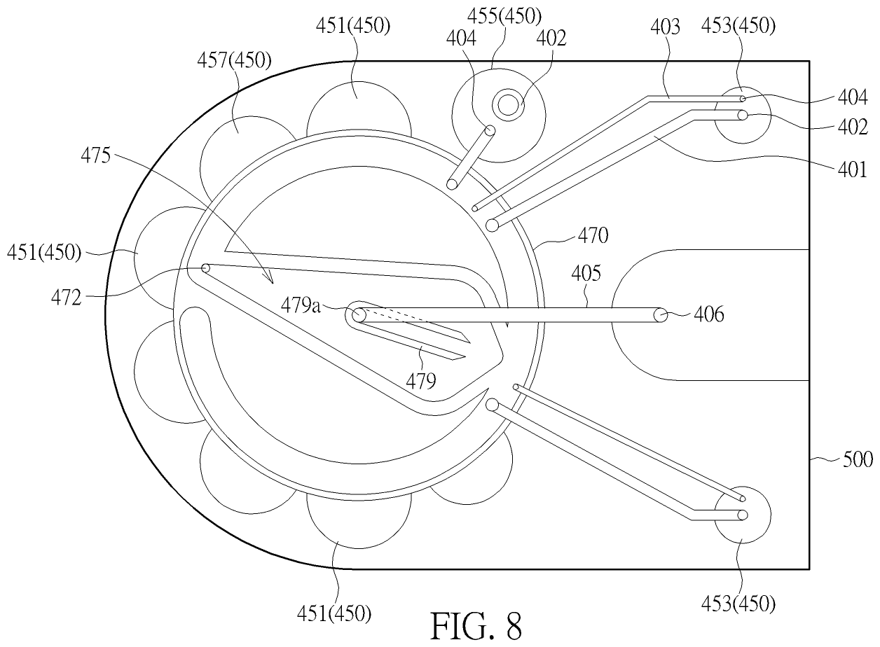

Please refers to FIG. 7 to FIG. 10, which illustrate an analysis cartridge 500 according to the second embodiment of the present disclosure, wherein FIG. 7 is a schematic diagram of an exploded view of the analysis cartridge 500, FIG. 8 is a schematic diagram of a top view of the analysis cartridge 500, and the rest are schematic diagrams of a stereo view or a cross-sectional view of the detailed components of the analysis cartridge 500. As shown in FIG. 7 and FIG. 8, the analysis cartridge 500 also includes a first cover 400, a second cover 410, a sealing layer 480 and a rotary valve 470, and the first cover 400 and the second cover 410 are separately from each other before assembling, so as to together define an accommodation space 460 therebetween. The structure, material selection and the assembling method of the analysis cartridge 500 in the present embodiment are all substantially the same as those of the analysis cartridge 300 in the first embodiment, and which will not be redundantly described hereinafter. The differences between the present embodiment and the first embodiment lie in that a third cover 430 is additionally disposed between the first cover 400 and the second cover 410, and the rotary valve 470 is rotably disposed on the third cover 430 and within the accommodation space 460 between the first cover 400 and the second cover 410. The first cover 400, the third cover 430 and the second cover 410 are assembled through a thermal melting method or an ultrasonic method, so as to sandwich the rotary valve 470 between the first cover 400 and the third cover 430 (as shown in FIG. 8), thereby improving the reliability and malleability of the analysis cartridge 500.

Precisely speaking, the first cover 400 and the second cover 410 also include mutually corresponding contours, such as the arch shape as shown in FIG. 7 to FIG. 8, but are not limited thereto. The first cover 400 further includes a plurality of fluid tunnels 401 and a plurality of gas tunnels 403 disposed thereon, wherein each of the fluid tunnels 401 and each of the gas tunnels 403 for example horizontally extend in any direction parallel to the direction D1 to connect to a pipette 402 or an gas hole 404, for fluid or gas circulation. On the other hand, the second cover 410 further includes a plurality of through holes 411 disposed thereon, and the through holes 411 may penetrate through the second cover 410 to accommodate a plurality of containers 450. In the present embodiment, although the sizes of each container 450 and each through hole 411 (for example, the diameter or the aperture of the container 450 and the through hole 411) are uniform, the practical arrangement is not limited thereto. In another embodiment, the arrangement of the through holes and the containers may also optionally include various sizes as reference to the through holes 111, 113, 115 and the containers 151, 153, 155 in the first embodiment. The containers 450 for example include a plurality of reagent container 451, at least one reaction container 453 and a least one sample container 455, wherein each of the reagent containers 451 may accommodate a cleaning reagent, a buffer, an eluent, a lysis buffer or the like, the at least one reaction container 453 may accommodate various enzymes or reactants (such as primers or probes) for performing the reaction, and the at least one sample container 455 may accommodate various samples such as bacteria, cells or virus or the samples suspected to contain bacteria, cells or viruses for performing the nucleic acid extraction and the nucleic acid amplification. Also, the containers 450 may further include an extraction container 457 having a plurality of magnetic beads (not shown in the drawings) disposed therein, and the magnetic beads may be combined with the testing sample for purification at the beginning of the test. In addition, it is noted that, the detailed features (such as the material selections, the structures or the arrangements) of the first cover 400, the second cover 410 and other components (such as the fluid tunnels 401, the pipettes 402, the gas tunnels 403, the gas holes 404, the containers 450 and the flat film material attached on the surface of the first cover 400) are all substantially the same as those in the first embodiment, and which will not be redundantly described hereinafter.

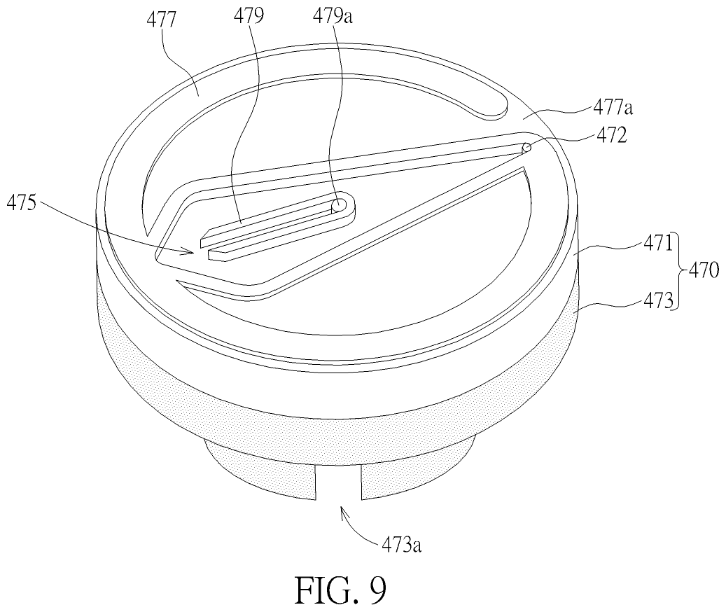

The rotary valve 470 of the present embodiment is also consisted of a soft material in combined with a hard material, in order to improve the airtightness of the rotary valve 470 after being combined with the first cover 400, the third cover 430 and the second cover 410. As shown in FIG. 9, the rotary valve 470 includes a first portion 471 and a second portion 473 stacked from top to bottom, wherein the second portion 473 for example includes a rigid material which is different from that of the first portion 471. The specific materials of the first portion 471 and the second portion 473 are substantially the same as those of the first portion 131 and the second portion 133 in the first embodiment, and it will not be redundantly described hereinafter. The first portion 471 further includes a protrusion 477, which surrounds the top surface of the first portion 471 to form a flow channel 475 and an opening 477a, and the second portion 473 of the rotary valve 470 includes an engagement 473a. In this way, after the analysis cartridge 500 is assembled, the first portion 471 of the rotary valve 470 may also attach to the first cover 400, and the second portion 473 of the rotary valve 470 may be protruded into the through hole 413, thereby achieving an airtight assemble manner. With such arrangement, the engagement 473a of the second portion 473 of the rotary valve 470 may externally connect to a motor (not shown in the drawings), with the motor driving and controlling the rotary valve 470 within the analysis cartridge 500 to rotate.

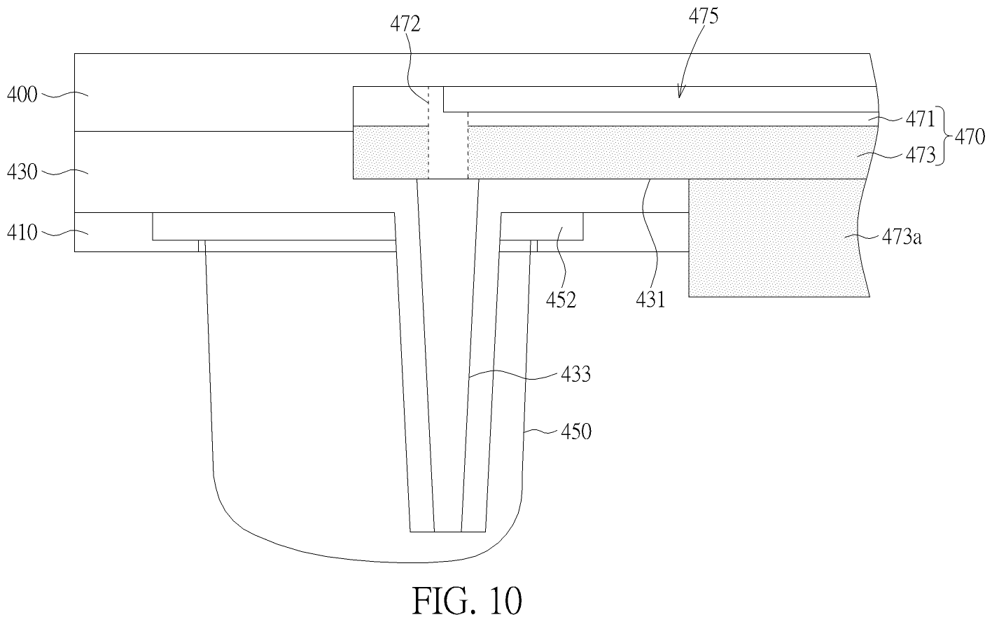

The difference between the present embodiment and the aforementioned embodiments is mainly in that the coverage area of the rotary valve 470 is greater than that of the rotary valve 130 in the aforementioned embodiments. For example, while observing a top view shown in FIG. 8, the rotary valve 470 may partially cover a part of the containers 450 disposed below, and in comparison, the rotary valve 130 in the aforementioned embodiments will not cover any container 150 (as shown in FIG. 2). Please also refer to FIG. 7 and FIG. 10, the rotary valve 470 is disposed on a base 431 of the third cover 430, and the coverage area of the base 431 may also partially cover a part of the containers 450. Furthermore, a plurality of pipettes 433 is disposed below the base 431, and each pipette 433 is in alignment with each container 450 underneath. While the analysis cartridge 500 is assembled, each of the pipettes 433 may penetrate through a film 452 on each container 450 to extend into each container 450. Precisely speaking, each of the pipettes 433 includes a hollow structure which is extended downwardly from the third cover 430 and protruded from a surface of the third cover 430. In the present embodiment, although the bottom of each pipette 433 is illustrated as a plane as shown in FIG. 10, the practical arrangement is not limited thereto. In another embodiment, the bottom of the pipettes may include an inclined sidewall as reference to the pipettes 102 of the aforementioned embodiments, so as to improve the problem that the pipettes are easy to remain in the pipettes when sucking liquid.

On the other hand, due to the expanded coverage area of the rotary valve 470, the flow channel 475 disposed on the rotary valve 470 may also have a larger volume accordingly, so as to accommodate more fluid. The flow channel 475 may include any suitable shape, such as a spindle shape as shown in FIG. 9, but is not limited thereto. It is noted that, the rotary valve 470 further includes a vertical channel 472 disposed thereon, and the vertical channel 472 penetrates through the first portion 471 and the second portion 473 of the rotary valve 470 to communicate with the flow channel 475 (as shown in FIG. 9 to FIG. 10). With such arrangements, the vertical channel 472 is allowable to be connected with each of the pipettes 433 in sequence by the rotation of the rotary valve 470. Then, while the rotary valve 470 is externally connected with a pump (not shown in the drawings) through its engagement 473a, various reagents within each container 450 may be sucked out, discharged, or transferred through a positive or a negative pressure supplied by the pump. Furthermore, in the present embodiment, the first portion 471 of the rotary valve 470 further includes a protruding ring 479 disposed around an air hole 479a. While the rotary valve 470 is used to suck out, discharge, or transfer various reagents through the assist of the pump, the air hole 479a disposed on the rotary valve 470 may be connected to a vent 406 through an air-guided channel 405 additionally disposed on the first cover 400, so that, the various reagents maybe fluently sucked out, discharged, or transferred.

Through these arrangements, the analysis cartridge 500 of the second embodiment in the present disclosure is provided. The analysis cartridge 500 may also freely transfer and mix the various fluids such as the samples, the reagents and the reactants within the containers 450 by using the rotary valve 470 disposed within the analysis cartridge 500, to carry out the detection reaction in the reaction container 453 finally. In this way, the analysis cartridge 500 may effectively provide an automated testing process of sample-in result-out. In the present embodiment, the coverage area of the rotary valve 470 is expanded, so that the rotary valve 470 may enable to partially cover the containers 450 underneath, and the flow channel 475 of the rotary valve 470 may also have an expanded volume correspondingly. Accordingly, while the external motor is linked with the rotary valve 470 disposed within the analysis cartridge 500 to drive the rotary valve 470 to rotate, the vertical channel 472 disposed on the rotary valve 470 may be directly aligned and communicated with the pipettes 433 penetrated into the containers 450, and the fluids may be sucked out and temporarily stored in the flow channel 475. Therefore, the fluid circulation path may be shortened, and the required time for the fluid to be sucked out, discharged or transferred may also be reduced significantly. Also, with these arrangements, the analysis cartridge 500 in the present embodiment may also obtain the simplified component configuration, in which, not only the liquid temporary storage region 170 of the aforementioned embodiments may be omitted, but also the specific number of the fluid tunnels 401 and/or the gas tunnels 403 disposed on the first cover 400 may be dramatically reduced. Thus, in comparison with the analysis cartridge 300 in the aforementioned embodiments, the analysis cartridge 500 may therefore gain more optimized testing efficiency and more simplified configuration, so as to meet the practical requirements of the testing products.

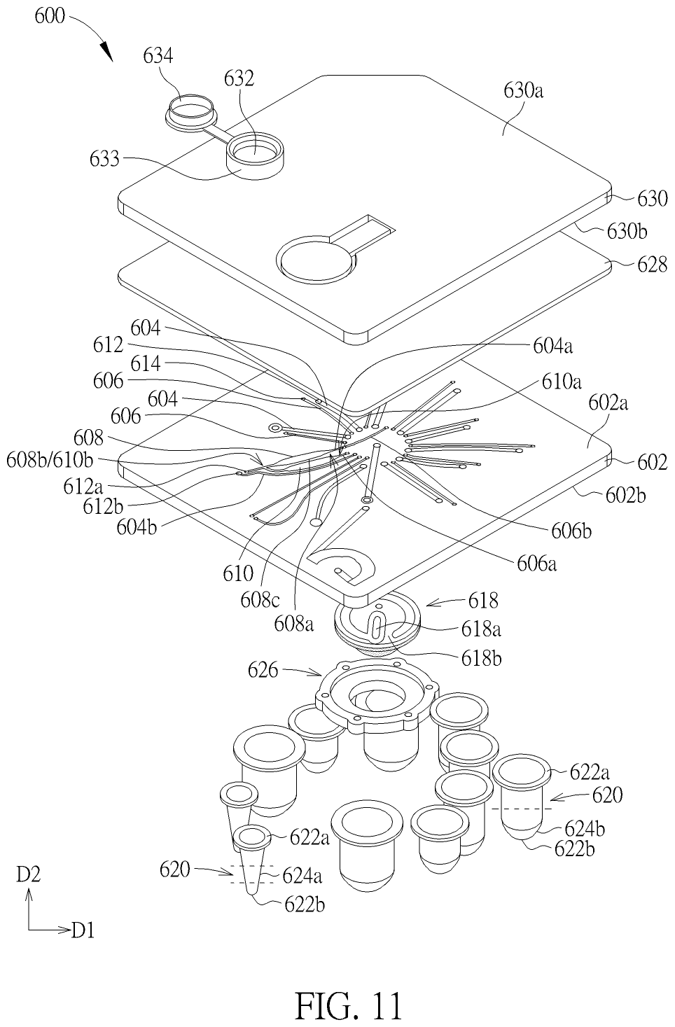

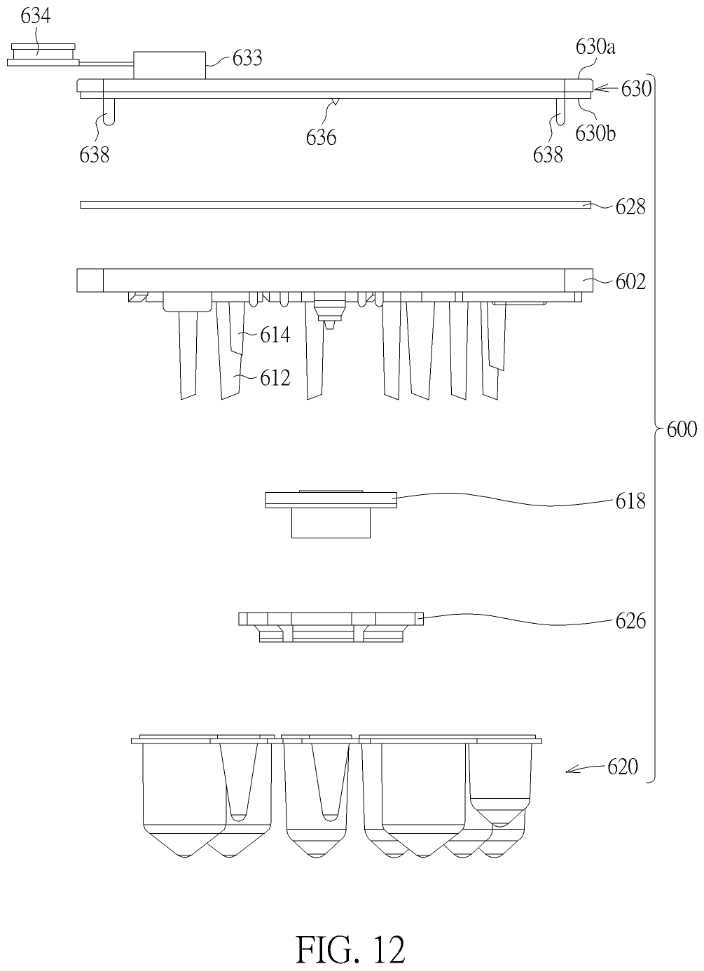

Please refer to FIG. 11 to FIG. 13, which illustrate an analysis cartridge 600 according to the third embodiment of the present disclosure. The structure and the components of the analysis cartridge 600 are substantially similar to those of the analysis cartridge 300 in the aforementioned embodiment, and all the similarities will not be redundantly described hereinafter. The different between the present embodiment and the aforementioned embodiments is mainly in that the analysis cartridge 600 further includes at least one quantification chamber.

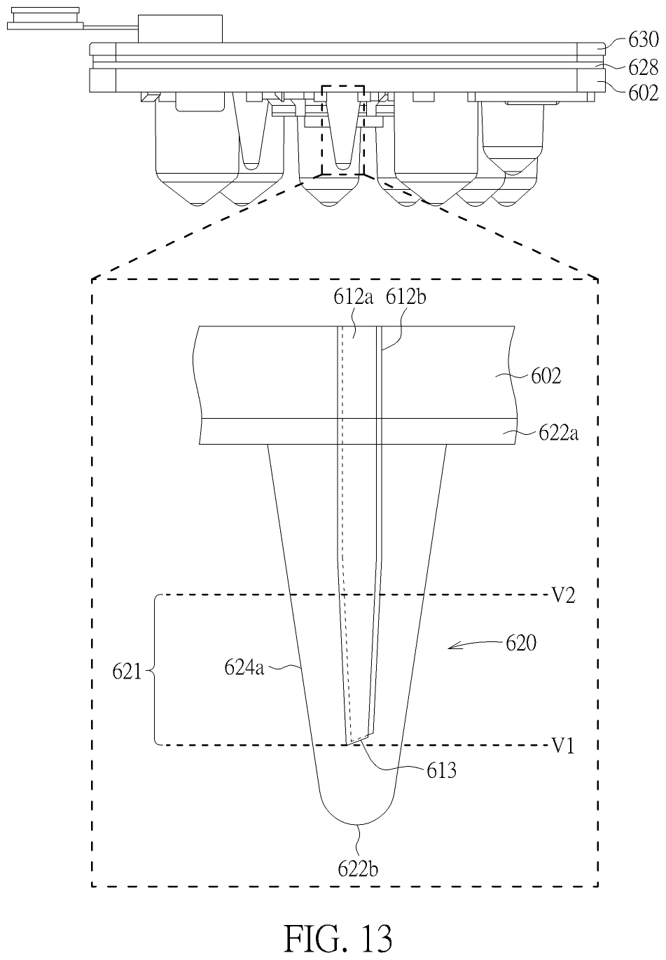

As shown in FIG. 11 to FIG. 13, the analysis cartridge 600 includes a main cover 602, at least one container 620, a first pipette 612a, and a rotatory valve 618. The main cover 602 has a first surface 602a and second surface 602b opposite to each other, and the first surface 602a further includes a first quantification chamber 608, a first fluid tunnel 604a, a first gas tunnels 606a and a storage chamber 610 disposed thereon, with the first quantification chamber 608, the first fluid tunnel 604a, the first gas tunnels 606a and the storage chamber 610 respectively extending in different horizontal directions. The first quantification chamber 608 has a first end 608a connected to the first fluid tunnel 604a, and a second end 608b connected to a second end 610b of the storage chamber 610 and the first pipette 612a. On the other hand, the storage chamber 610 has a first end 610a connected to the first gas tunnel 606a. The container 620 is disposed on the second surface 602b of the main cover 602, with the container 620 disposed right below the second end 608b of the first quantification chamber 608 in a vertical direction D2 (such as the y-direction in FIG. 11). The first pipette 612a is disposed on the main cover 602, with the first pipette 612a having a hollow structure extended downwardly from the first surface 602a to protrude from the second surface 602b in the vertical direction D2. The first pipette 612a is disposed within the container 620 (as shown in FIG. 13), wherein the first pipette 612a is connected to the second end 608b of the first quantification chamber 608 and the first fluid tunnel 604a. The rotary valve 618 is rotably disposed on the second surface 602b, and which includes a flow tunnel 618a and an opening 618b disposed thereon, to respectively connect the first fluid tunnel 604a and the first gas tunnel 606a through rotating the rotary valve 618. It is noteworthy that, the first quantification chamber 608 disposed within the analysis cartridge 600 enables to initiatively quantify a reagent when injecting the reagent into the container 620, so as to dramatically improve the analysis sensitivity and the efficiency of an analysis method which is conducted in the analysis cartridge 600.

Precisely speaking, the first quantification chamber 608 and the storage chamber 610 for example respectively include a space recessed from the first surface 602 for containing and quantifying a required reagent, and the space may include any shape and volume based on practical requirements. In one embodiment, the first quantification chamber 608 preferably includes two inclined sidewalls 608c as shown in FIG. 11, with the two inclined sidewalls 608c disposed at two opposite sides of the first quantification chamber 608 in the horizontal direction to avoid the reagent remained in the first quantification chamber 608 while injecting the required reagent into the container 620. Otherwise, in another embodiment, the first quantification chamber 608 may further include a hydrophobic membrane (not shown in the drawings) coating on the inclined sidewalls 608c of the first quantification chamber 608, for further facilitating the absorbing and the injecting of the required reagent.

Further in view of FIG. 11, the main cover 602 further includes a second gas tunnel 606b extending in the same horizontal direction of the first fluid tunnel 604a. The second gas tunnel 606b is connected to the first fluid tunnel 604a, and which enables to further connect to the opening 618b through rotating the rotary valve 618. The main cover 602 further includes a second fluid tunnel 604b and a second pipette 612b, with the second fluid tunnel 604b extending on the first surface 602a of the main cover 602 along another horizontal direction, and with the second pipette 612b also having a hollow structure extended downwardly from the first surface 602a to protrude from the second surface 602b in the vertical direction D2. It is noted that the second fluid tunnel 604b is extended to connect the second pipette 612b, and the second pipette 612b is also disposed within the container 620 (as shown in FIG. 13), wherein the second fluid tunnel 604b enables to aligned with the flow channel 618a through rotating the rotary valve 618.

The container 620 is mounted to the second surface 602b through a plurality through holes (not shown in the drawings) disposed on the main cover 602. As shown in FIG. 13, the container 620 further includes a second quantification chamber 621 disposed therein, between a top 622a and a bottom 622b of the container 620. The second quantification chamber 621 is a buffering region within the container 620, wherein a bottom surface V1 of the second quantification chamber 621 and the bottoms of the first pipette 612a and the second pipette 612b are preferably on the same plane, and a top surface V2 of the second quantification chamber 621 is lower than the top surfaces of the first pipette 612a and the second pipette 612b. It is noted that each of the first pipette 612a and the second pipette 612b has an inclined bottom surface 613, for easily extending into the container 620, wherein the first pipette 612a and the second pipette 612b are extended to not directly contacting the bottom 622b of the container 620, as shown in FIG. 13.

In one embodiment, the container 620 preferably includes an inclined sidewall 624a in the vertical direction D2, at two sides thereof, and the inclined sidewalls 624a may be extended over the whole region of the second quantification chamber 621 as shown in FIG. 13, to avoid the required reagent remained in the container 620 while absorbing the reagent from the container 620. In another embodiment, the inclined sidewall 624b may also be extended only within a partial region of the second quantification chamber 621, as shown in FIG. 11.

The analysis cartridge 600 further includes a sealing layer 628 and a package cover 630, the sealing layer 628 is disposed on the first surface 602a of the main cover 602 to seal the fluid tunnels 604, the gas tunnels 606, the first quantification chamber 608 and the storage chamber 610 on the main cover 602, and the package cover 630 is disposed on the sealing layer 628. The package cover 630 further includes a first surface 630a and a second surface 630b opposite to the first surface 360a, wherein the first surface 630a includes a sealing film 632, an airtight ring 633 and a container cover 634 disposed thereon, with the airtight ring 633 being aligned with the container 620 underneath and with the sealing film 632 sealing the container 620, and the container cover 634 is disposed on the airtight ring 633 to cover the container 620 underneath. The second surface 630b includes at least one pin 636 and a plurality of assemble pillars 638, wherein the pin 636 is disposed on the package cover 630 for penetrating through the sealing layer 628 after assembling the package cover 630 and the main cover 602, and the assemble pillars 638 are disposed around the outer periphery of the package cover 630 for facilitating the assembling of the package cover 630 and the main cover 602. In this way, the package cover 630 can be attached to the sealing layer 628 and the main cover 602 through the assemble pillar 638 via a suitable bonding process, but not limited thereto.

Through these arrangements, while the rotary valve 618 further connect to an external pump (not shown in the drawings), the required reagent may be transferred to the contain 620 via the first quantification chamber 608, to precisely quantify the volume of the required reagent. It is noted that, while the flow tunnel 618a of the rotary valve 618 is aligned with the first fluid tunnel 604a connected to the first end 608a of the first quantification chamber 608, and the opening 618a of the rotary valve 618 is aligned with the first gas tunnel 606a connected to the first end 610a of the storage chamber 610, the required reagent is firstly injected into the first quantification chamber 608 through the first end 608a of the first quantification chamber 608 for primary quantification, and a redundant portion of the redundant reagent is next transferred to the storage chamber 610 through the second end 608b of the first quantification chamber 608 (the second end 610b of the storage chamber 610) due to the pressure difference between the storage chamber 610 and the first quantification chamber 608. Then, the opening 618a of the rotary valve 618 is aligned with the second gas tunnel 606b connected to the first fluid tunnel 604a by rotating the rotary valve 618, and the quantified reagent within the first quantification chamber 608 can be pushed by air-pressure and injected into the container 620 through the pipette 612 connected to the second end 608b of the first quantification chamber 608. Meanwhile, since the storage chamber 610 is not connected to any fluid tunnel 604, the redundant portion of the reagent will be remained in the storage chamber 610, without flowing into the container 620. In this way, the reagent injected into the container 620 may be initiatively quantify through the first quantification chamber 608, instead of being passively quantify through the external pump, and the required reagent with precisely controlled volume may be next mixed with a froze-dry sample accommodated in the container 620 to conduct a required reaction. In addition, while absorbing the mixed reagent from the container 620, the flow channel 618a of the rotary valve 618 is aligned with the second fluid tunnel 604b connected to the second pipette 612b by rotating the rotary valve 618 again, and only the mixed reagent within the second quantification chamber 621 is sucked out via the second pipette 612b and the second fluid tunnel 604b, with the mixed reagent below the second quantification chamber 621 being remained in the container 620. Accordingly, the sucked-out volume (V2-V1) of the mixed reagent may be accurately controlled. Thus, the analysis cartridge 600 of the present embodiment is allowable to initiatively quantify the reagent either when absorbing the reagent from a container 620, or when injecting the reagent into the container 620, and which can be applied on the reaction with strict quantitative requirements such as a nucleic acid extraction or a nucleic acid amplification, to dramatically increase the reaction efficiency and the sensitivity.

In one embodiment, the analysis cartridge 600 may further includes a plurality of fluid tunnels 604 and a plurality of gas tunnels 606 disposed on the first surface 602a of the main cover 602, a plurality of the containers 620 disposed on the second surface 602b of the main cover 602, and a plurality of pipettes 612 and a plurality of air pipes 614 respectively extended downwardly from the first surface 602a to protrude from the second surface 602b, as shown in FIG. 11 to FIG. 13. The fluid tunnels 604 and the gas tunnels 606 are extended from the center to the periphery of the main cover 602, with each of the fluid tunnels 604 respectively connected to each of the pipettes 612, and with each of the gas tunnels 606 respectively connected to each of the air pipes 614. It is noted that each of the pipettes 612 and each of the air pipes 614 are aligned with a corresponding one of the containers 620, to extend into the corresponding one of the containers 620. It is also noted that, each of the air pipe 614 and the pipette 612 also includes a hollow structure and an inclined bottom surface (not shown in the drawings) for easily extending into the corresponding one of the containers 620. The containers 620 may include a sample container, an extraction container, a reaction container, a reagent container, at least one washing container and any required container due to practical requirements, and which are respectively attached to the main cover 602, and the rotary valve 618 is also attached to the second surface 602b of the main cover 602 through abase 626 disposed below the rotary valve 618, but not limited thereto. People in the art should fully realize that the number, the size and the shape of the container 620 are all not limited to what is shown in FIG. 11, and which can be further adjustable based on practical product requirements.

In a preferably embodiment of an analysis method by using the analysis cartridge 600 for example for conducting a nucleic acid extraction and a nucleic acid amplification, a sample within the sample container is firstly transferred to the extraction container through the rotary valve 618, for breaking the cell within the sample and releasing the nucleic acid from the cell, and then capturing the nucleic acid by magnetic beads within the extraction container. Next, the nucleic acid captured by the magnetic beads is sequentially transferred to each washing container through the rotary valve 618 for repeatedly cleaning the nucleic acid. Then, the nucleic acid is eluted from the magnetic beads and transferred to the reagent container. It is noted that the nucleic acid is transferred to the reagent container via the rotary valve 618 and the first quantification chamber 608, for precisely controlling the volume of the nucleic acid to mix with the froze-dry sample within the reagent container. Then, a mix of the nucleic acid and the froze-dry sample are further transferred to the reaction container, with the mix being quantified through the second quantification chamber 621, and an accurate volume of the mix is allowable to carried out a required reaction, such as nucleic acid amplification, under precise-controlled temperature conditions. In other words, through the first quantification chamber 608 and/or the second quantification chamber 621 additionally disposed in the analysis cartridge 600, the reagent and the sample within the analysis cartridge 600 enable to be initiatively quantified either when absorbing the reagent or the sample from the containers 620, or when injecting the reagent or the sample into the containers 620.

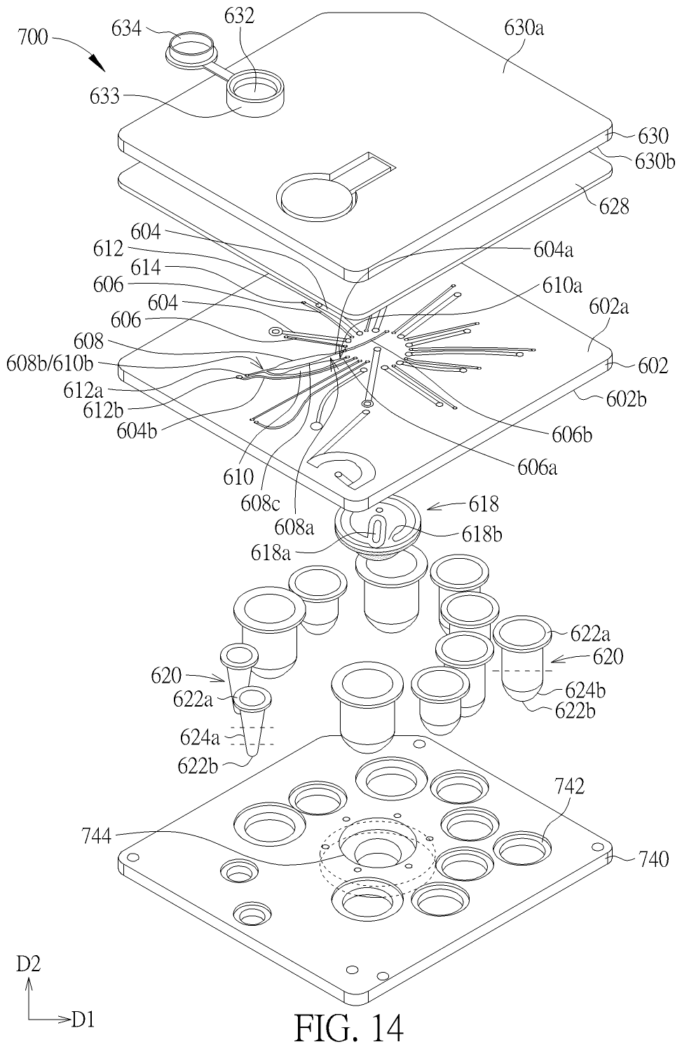

Please refer to FIG. 14, which illustrate an analysis cartridge 700 according to the fourth embodiment of the present disclosure. The structure and the components of the analysis cartridge 700 are substantially similar to those of the analysis cartridge 600 in the aforementioned embodiments, and all the similarities will not be redundantly described hereinafter. The different between the present embodiment and the aforementioned embodiment is mainly in that the analysis cartridge 700 further includes a bottom cover for carrying the containers 620 and the rotary valve 618.

Precisely speaking, as shown in FIG. 14, the analysis cartridge 700 further includes a bottom cover 740 attached to the second surface 602b of the main cover 602. The bottom cover 740 includes a plurality of through holes 742, 744, with each of the through holes 742, 744 penetrating through two opposite surfaces of the bottom. cover 740. It is noted that each of the through holes 742, 744 may include various size for accommodating the containers 620 and the rotary valve 618 respectively. In this way, the container 620 and the rotary valve 618 will be sandwiched between the main cover 602 and the bottom cover to gain better structural reliability.

In summary, the present disclosure provides an analysis cartridge, which is assembled by two or more than two covers via a thermal melting method or an ultrasonic method. The analysis cartridge includes the rotary valve which is ratably disposed therein, with the rotary valve being rotated by being linked with an external motor to form the fluid circulation paths like a “container-fluid tunnel-flow channel on the rotary valve-fluid tunnel-container” path, a “container-fluid tunnel-flow channel on the rotary valve-liquid temporary storage region-fluid tunnel-container” path, or a “container-vertical channel on the rotary valve-flow channel on the rotary valve-container” path. Also, the analysis cartridge further includes the quantification chamber enables to initiatively quantify a reagent either when absorbing the reagent from the container, or when injecting the reagent into the container, instead of being passively quantify through the external pump. Therefore, the various reagents within each container in the analysis cartridge may be successfully and precisely sucked out, discharged, transferred, and mixed through a positive pressure or a negative pressure supplied by the pump, and finally to carry out a predetermined detection reaction such as a nucleic acid amplification, a probe binding reaction or an enzyme binding reaction in a reaction container.

Then, the analysis cartridge of the present disclosure may achieve an automated testing process of sample-in result-out. Besides, people in the art should fully understand that, the analysis cartridge not only may be used in nucleic acid extraction and nucleic acid testing, but also maybe further in used in other testing fields based on practical requirements. For example, in other embodiments, the analysis cartridge of the present disclosure may also be used in protein sample extraction and enzyme immune reaction.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An analysis cartridge, comprising:
a main cover, having a first surface and a second surface opposite with each other, the first surface comprising a first quantification chamber, a first fluid tunnel, a first gas tunnel and a storage chamber extending along horizontal directions, wherein a first end of the first quantification chamber is connected to the first fluid tunnel, a first end of the storage chamber is connected to the first gas tunnel, and a second end of the first quantification chamber is connected to a second end of the storage chamber;
at least one container, disposed on the second surface of the main cover, the at least one container overlapped the second end of the first quantification chamber in a vertical direction;
a first pipette, disposed on the main cover and protruded from the second surface of the main cover, wherein the first pipette is connected to the second end of the first quantification chamber and disposed within the at least one container; and
a rotary valve, rotably disposed on the second surface of the main cover.

2. The analysis cartridge according to claim 1, wherein the at least one container comprises a second quantification chamber disposed therein, the second quantification chamber is disposed between a top and a bottom of the at least one container, and a bottom surface of the second quantification chamber and a bottom of the first pipette are in a same plane.

3. The analysis cartridge according to claim 2, wherein the second quantification chamber comprises an inclined sidewall.

4. The analysis cartridge according to claim 1, wherein the rotary valve further comprises a flow channel and an opening, the flow channel and the opening are aligned with the first fluid tunnel and the first gas tunnel respectively.

5. The analysis cartridge according to claim 4, wherein the first surface of the main cover further comprises:
a second gas tunnel connected to the first fluid tunnel, wherein the opening is aligned with the second gas tunnel.

6. The analysis cartridge according to claim 4, wherein the main cover further comprises:
a second fluid tunnel extending on the first surface of the main cover, wherein the flow channel is aligned with the second fluid tunnel; and
a second pipette disposed on the main cover and protruded from the second surface of the main cover, wherein the second pipette is connected to the second fluid tunnel and disposed within the at least one container.

7. The analysis cartridge according to claim 1, further comprising:
a sealing layer, disposed on the first surface of the main cover; and
a package cover, disposed on the sealing layer.

8. The analysis cartridge according to claim 7, wherein the package cover further comprises:
an airtight ring, disposed on a first surface of the package cover and aligned with the at least one container;
a container cover, disposed on the airtight ring and covering the at least one container; and
at least one pin, disposed on a second surface of the package cover.

9. The analysis cartridge according to claim 7, wherein the package cover further comprises a sealing film to seal the at least one container.

10. analysis cartridge according to claim 1, further comprising:
a plurality of fluid tunnels and a plurality of gas tunnels, disposed on the first surface of the main cover and extended along different horizontal directions respectively;
a plurality of pipettes and a plurality of air pipes, disposed on the main cover and respectively protruded from the second surface of the main cover in the vertical direction, wherein each of the pipettes is connected to each of the fluid tunnels, and each of the air pipes is connected to each of the gas tunnels; and
a plurality of the containers, respectively disposed on the second surface of the main cover, wherein each one of the air pipes and each one of the pipettes are disposed within one of the containers.

11. An analysis method, comprising:
providing an analysis cartridge comprising:
a main cover comprising a first quantification chamber, a first fluid tunnel, a first gas tunnel and a storage chamber extending along different horizontal directions, wherein a first end of the first quantification chamber is connected to the first fluid tunnel, a first end of the storage chamber is connected to the first gas tunnel, and a second end of the first quantification chamber is connected to a second end of the storage chamber;
a first pipette disposed on the main cover, wherein the first pipette is connected to the second end of the first quantification chamber;
a plurality of containers disposed on the main cover, wherein one of the containers is overlapped the second end of the first quantification chamber in a vertical direction and comprises a froze-dry sample disposed therein; and
a rotary valve, rotably disposed on the main cover;
injecting a reagent into the first quantification chamber through the first end of the first quantification chamber;
after injecting the reagent into the first quantification chamber, transferring a portion of the reagent into the storage chamber through the second end of the first quantification chamber; and
transferring the reagent within the first quantification chamber into the one of the containers through the rotary valve, to mix the froze-dry sample within the one of the containers.

12. The analysis method according to claim 11, before injecting a reagent into the first quantification chamber, further comprising:
rotating the rotary valve till a flow channel of the rotary valve being align with the first fluid tunnel, and an opening of the rotary valve being aligned with the second fluid tunnel.

13. The analysis method according to claim 12, wherein the main cover further comprises:
a second gas tunnel connected to the first fluid tunnel;
a second fluid tunnel extending on the first surface of the main cover; and
a second pipette disposed on the main cover and connected to the second fluid tunnel, wherein the second pipette is disposed within the one of the containers, and the one of the containers further comprises a second quantification chamber disposed therein, and the second quantification chamber is disposed between a top and a bottom of the one of the containers.

14. The analysis method according to claim 13, before transferring the reagent within the first quantification chamber into the one of the containers, further comprising:
rotating the rotary valve till the opening of the rotary valve being align with the second gas tunnel.

15. The analysis method according to claim 13, further comprising:
absorbing a mix of the froze-dry sample and the reagent from the one of the containers through the second quantification chamber and the second pipette; and
transferring the mix of the froze-dry sample and the reagent to another one of the containers through the second fluid tunnel.

16. The analysis method according to claim 15, before absorbing a mix of the froze-dry sample and the reagent from the one of the containers, further comprising:
rotating the rotary valve till the flow channel of the rotary valve being align with the second fluid tunnel.

17. The analysis method according to claim 13, wherein a bottom surface of the second quantification chamber and a bottom of the first pipette are in a same plane.

18. The analysis method according to claim 13, wherein a top surface of the second quantification chamber is lower than the top of the one of the containers.

19. The analysis method according to claim 11, before injecting the reagent into the first quantification chamber further comprising:
transferring a sample to another container through the rotary valve;
extracting nucleic acid from the sample;
transferring the nucleic acid to other containers sequentially through the rotary valve; and
washing the nucleic acid in the other containers, to obtain the reagent.

20. The analysis method according to claim 19, wherein the another container further comprises magnetic beads disposed therein, and the nucleic acid is captured by the magnetic beads.
